(19) United States
(12) Patent Application Publication
(10) Pub. No.: US 2026/0325759 A1
(43) Pub. Date: Oct. 22, 2026

(54) SYRINGE ATTACHMENT FOR ONE-HANDED INJECTION

(71) Applicants: Shaare Zedek Scientific Ltd., Jerusalem (IL); Azrieli College of Engineering Jerusalem, Jerusalem (IL)

(72) Inventors: Haytam Kasem, Iksal (IL); Israel Dvir, Beni-Brak (IL); Kareem Abd Rbo, Jerusalem (IL); Emmanuel Bettach, Tel Aviv (IL)

(73) Assignees: Shaare Zedek Scientific Ltd., Jerusalem (IL); Azrieli College of Engineering Jerusalem, Jerusalem (IL)

(21) Appl. No.: 19/104,709

(22) PCT Filed: Aug. 15, 2023
(86) PCT No.: PCT/IL2023/050863

§ 371 Date: Feb. 19, 2025

Related U.S. Application Data
(60) Provisional application No. 63/401,722, filed on Aug. 29, 2022.

(51) Int. Cl. A61M 5/315; A61M 5/31

(52) U.S. Cl. CPC A61M 5/31501; A61M 5/31566; A61M 5/3158; A61M 2005/3143; A61M 2005/31508; A61M 2205/0216; A61M 2205/3331; A61M 2205/3379; A61M 2205/586

(57) ABSTRACT

The present invention relates to a syringe attachment device and/or a method for facilitating one-handed controlled of the volume and/or rate of injection of an existing syringe.

### RELATED APPLICATION/S

This application claims the benefit of priority under 35 USC § 119(e) of U.S. Provisional Patent Application No. 63/401,722 filed 29 Aug. 2022, the contents of which are incorporated herein by reference in their entirety.

### FIELD AND BACKGROUND OF THE INVENTION

The present invention in some embodiments thereof relates to a syringe attachment for one-handed injection.

When treating patients during microsurgical procedures, the surgeons are often required to inject a variety of substances into sensitive tissues. These injections are performed on very sensitive human tissues like intraocular tissue, often through minimal surgical incisions and/or with very thin needles or cannulas. For example, ophthalmologists may need to inject a variety of substances into the eye. These substances include low viscosity drugs, high viscosity liquids, hydrogels, and even semi-solid objects e.g., injectable intraocular lenses (IOLs).

Therefore, users desire precise needle and syringe manual control (to fit a pre-existing small incision and/or avoid clumsy movement which could accidentally damage tissue), the ability to apply a relatively high force to the plunger to achieve controlled sliding without causing movement of the needle, precise control of injection rate and volume. Currently, to optimize the control of the syringe and the needle, the surgeon generally uses two hands, one for holding the syringe proximally (the side closest to the tissue being treated; in general, the outlet of the syringe and/or the needle of the syringe are located on the proximal end thereof), and the other to push the plunger. In this disclosure, the proximal side of the syringe and/or attachment device will also be referred to as the front.

Another issue is that many of these drugs/devices are pre-loaded into syringes. Therefore, there is a need for a device facilitating injection of a substance with high control of the syringe position and rate of injection by the surgeon with one hand.

### SUMMARY OF THE INVENTION

According to an aspect of some embodiments of the invention, there is provided a system for one-handed injection including: a syringe including a plunger and a barrel; and a syringe attachment device, wherein the syringe is coupled to the syringe attachment device; and wherein the syringe attachment device includes: a syringe interface configured to grip the barrel of the syringe; a main actuator configured to apply force between the syringe interface and the plunger; a braking system configured for inhibiting movement of the plunger; a trigger configured to graduate activation of the braking system; wherein the system is configured for simultaneously controlling a needle position and for actuating the trigger with one hand.

According to some embodiments of the invention, the system further includes a plunger interface interconnecting between the main actuator and the plunger and wherein the inhibiting movement of the plunger is by the braking system inhibiting movement of the plunger interface.

According to some embodiments of the invention, the plunger interface grasps the plunger.

According to some embodiments of the invention, the plunger interface grasps a flange of the plunger.

According to some embodiments of the invention, the syringe interface grasps a flange of the barrel of the syringe.

According to some embodiments of the invention, the braking system includes a static brake pad and at least one kinetic brake pad.

According to some embodiments of the invention, at least one of the static brake pads and the kinetic brake pad includes a micro-patterned brake pad.

According to some embodiments of the invention, at least one of the static brake pads and the kinetic brake pad is biomimetic.

According to some embodiments of the invention, the micro-patterned brake pad is composed at least in part by an elastomer.

According to some embodiments of the invention, the elastomer has an elastic modulus ranging between 3-10 MPa.

According to some embodiments of the invention, the elastomer includes poly(dimethylsiloxane) (PDMS), polydioxanone (PDO, PDS) or a combination thereof.

According to some embodiments of the invention, the braking system is biased to prevent limit movement of the plunger and wherein actuating the trigger releases the braking system in a graduated manner to facilitate graduated movement of the plunger with respect to the barrel.

According to some embodiments of the invention, a predefined maximum rate of injection is set by the trigger, the braking system, the main actuator or any combination thereof.

According to some embodiments of the invention, the trigger is configured to release the braking system permitting the main actuator to depress the plunger.

According to some embodiments of the invention, a braking force is controlled by the trigger.

According to some embodiments of the invention, the braking force is released gradually according to a displacement of the trigger to facilitate gradual increase in rate of movement of the plunger in response to gradual increase of displacement on the trigger.

According to some embodiments of the invention, the syringe interface is configured to grip a syringe barrel, a needle interface, or both.

According to some embodiments of the invention, the device is lightweight.

According to some embodiments of the invention, the device is disposable.

According to some embodiments of the invention, the device is easy to clean and to sterilize.

According to an aspect of some embodiments of the invention, there is provided a method of using a syringe attachment for one-handed injections, the method including: supplying a syringe in a syringe attachment device with a main actuator configured to apply a force between a plunger of the syringe and a barrel of the syringe and the attachment device further including a braking system inhibiting movement of the plunger with respect to the barrel; activating a trigger of the device thereby releasing a braking system resulting in discharge of the syringe.

According to some embodiments of the invention, the method further includes increasing a rate discharge of the syringe by increasing a level of activation of the trigger.

According to some embodiments of the invention, the method further includes simultaneously controlling a position of a needle of the syringe and the activating with one hand.

According to some embodiments of the invention, the method further includes simultaneously controlling a position of a needle of the syringe and the activating with one hand.

### DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a schematic view of a system in accordance with an embodiment of the current invention.

FIG. 2 is a schematic view of a system in accordance with an embodiment of the current invention.

FIG. 3 is a schematic view of a system in accordance with an embodiment of the current invention.

FIG. 4 is a schematic view of a Main Actuator of a system in accordance with an embodiment of the current invention.

FIG. 5 is a schematic view of a braking system in accordance with an embodiment of the current invention.

FIG. 6 is a schematic view of a braking system in accordance with an embodiment of the current invention.

FIG. 7 is a schematic view of a braking system in accordance with an embodiment of the current invention.

FIG. 8 a flow chart for a method of discharging contents of a syringe in accordance with an embodiment of the current invention.

FIG. 9 is a block diagram of an embodiment of the current invention.

FIG. 10 is a schematic view of a system in accordance with an alternative embodiment of the current invention.

FIG. 11 is a schematic view of a static brake pad in accordance with an embodiment of the current invention.

FIG. 12 is a graph indicating the superiority and advantage which may be gained from micropatterned surfaces in accordance with an embodiment of the current invention.

FIGS. 13A and 13B are various schematic views of an open and closed states of the system, respectively, in accordance with an embodiment of the current invention.

FIGS. 14A-14D are various schematic views of an open state system in accordance with an embodiment of the current invention.

FIGS. 15A-15C are various schematic views of an open state system in accordance with an embodiment of the current invention.

FIGS. 16A-16D are various schematic views of a closed state system in accordance with an embodiment of the current invention.

### DESCRIPTION OF SPECIFIC EMBODIMENTS OF THE INVENTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### Overview

The present invention in some embodiments thereof relates to a syringe attachment for one-handed injection and, more particularly, but not exclusively, to a syringe attachment for one-handed automatic injection. Optionally, the syringe attachment includes a biomimetic-based micro-pattern gripping brake pad, configured to provide a user with constant and/or gradual control of both the rate and/or volume of the injection.

In some embodiments, the device may fit onto a distal portion of an existing syringe (herein distal is used to refer to the end of the syringe away from patient. For example, the distal end is generally away from the discharge end and/or needle. For example, the plunger may enter the distal end of the barrel and/or a flange may be located on a distal portion of the barrel and/or a distal portion of a plunger. For example, discharge of the contents of a syringe may be driven by pushing a distal end of a plunger proximally). In this disclosure, the distal side of the syringe and/or attachment device will also be referred to as the back. In some embodiments, the device may apply pressure to a plunger. Optionally, a rate of injection may be under the control of the user. Optionally, the rate of injection may be constant. Optionally the rate of injection may be varied. Optionally, rate and/or volume of injection may be controlled by the user.

In some embodiments, the device may be lightweight e.g., between 10 to 50 g, and/or between 50 to 100 g, and/or between 100 to 200 g, and/or between 200 to 400 g, and/or between 400 to 800 g.

According to some embodiments, the movement of the device may be stable and/or controllable and/or predictable. In some embodiments, the movement may include applying relatively high forces to push highly viscous fluids or objects through a thin needle.

In some embodiments, the device may be inexpensive to produce. Optionally, the device may be disposable. In some embodiments, the device may be easy to clean and/or to sterilize.

According to some embodiments, the device may be compatible with one and/or a variety of existing syringe sizes and types. Optionally, the device may facilitate holding the syringe (e.g., controlling the needle position). Optionally, the device may facilitate controlling the rate and/or volume of injection with one hand. Optionally, the injection may be continuous. In some embodiments, the device may not interfere with the user's view of the movement of the plunger while gripping the proximal end of the syringe. Optionally, the device may be mechanical. Alternatively, and/or additionally, the device may include an electrical indicator and/or control and/or actuator.

Some embodiments relate to a syringe attachment device and/or a method for facilitating one-handed control of a volume and/or rate of injection of an existing syringe.

Optionally, the device may stably grip a distal portion of the syringe, e.g., within the distal most 1/10, and/or within the distal most ⅕, and/or within the distal most ½ of the syringe barrel and/or barrel flange. Optionally, the device may grip a distal portion of the syringe plunger, e.g., within the distal most 1/10, and/or within the distal most ⅕, and/or within the distalmost ½ of the syringe barrel and/or plunger flange. Alternatively, and/or additionally, the device may grip a proximal portion of the syringe barrel and/or plunger, e.g., within the proximal most 1/10, and/or within the proximal most ⅕, and/or within the proximal most ½ of the syringe barrel and/or barrel flange.

According to some embodiments, the device may include a source of force (e.g., an actuator) capable of exerting a reliable stable force on the plunger (e.g., between the plunger and the barrel of the syringe). Optionally, the source of force may be an elastic member, for example a spring and/or an elastic band, etc. Optionally, the source of force may be configured for controlled movement of the plunger with respect to the barrel of the syringe.

In some embodiments, the device may include a regulator (for example, a lever, a button, etc.). In some embodiments, the regulator may be controlled by the same hand of the user that is holding the proximal end of the syringe (e.g., by a finger or thumb of the user). Optionally, this may facilitate continuous control of flow rate. For example, flow rate may increase smoothly with increasing pressure on the trigger and/or with increasing displacement of the trigger. Optionally, the trigger that activates discharge of the syringe (e.g., by releasing a brake) is located and/or can be actuated from a proximal within the proximal most 1/10, and/or within the proximal most ⅕, and/or within the proximal most ½ of the syringe barrel.

In some embodiments, the device may facilitate viewing the syringe barrel and/or the contents thereof. For example, installing the device onto a syringe may leave the barrel, and/or a portion thereof uncovered, e.g., such that a user may see a proximal portion of the barrel from at least one side thereof. Alternatively, and/or additionally, the device may include a window through which the user can see the barrel of the syringe while performing an injection.

According to some embodiments, there may be various versions for the device. For example, there may be left hand and/or the right-hand version of the device, and/or there may be an ambidextrous version of the device. Alternatively, and/or additionally, the device may be symmetrical. In some embodiments, there may be different versions of the device suitable for various sizes and/or shapes of syringes and/or for various levels of force required to dispense the contents, and/or for different lengths of movement.

According to some embodiments, controls of the device may include a friction brake. Optionally, the friction brake may include a brake pad configured to facilitate continuous control of friction over a range of velocities (flow rates). For example, avoiding the slip-stick phenomenon. Optionally, the brake pads and/or other systems may include bionic components inspired by natural systems. Optionally, the brake pads may be made out of a micro-textured material e.g., based on the foot-pads of climbing frogs. Optionally, the brake pad may be elastomeric with micropatterns (for example, channels, micro-pillars). Optionally, the elastomeric material may be a soft elastomer. Optionally, the soft elastomer may be poly(dimethylsiloxane) (PDMS) and/or polydioxanone (PDO, PDS). Optionally, the soft elastomer may have an elastic modulus ranging between 3-10 MPa, and/or between 4-8 MPa, and/or between 5-7 MPa.

According to some embodiments, the syringe may be composed of glass, and/or plastic, and/or metal, and/or other materials, and/or any combination thereof. In some embodiments, the device may be loaded with a standard syringe, and/or a preloaded syringe. Optionally, the syringe may be loaded and/or pre-loaded with a drug containing liquid, a highly viscous liquid, an elastic and/or deformable gel, and/or an intraocular lens, or any combination thereof. Optionally, the syringe may have a capacity ranging between 0.2-0.5 ml, and/or between 0.5-0.8 ml, and/or between 0.8-1.3 ml, and/or between 1.3 to 10 ml.

According to some embodiments, the syringe may include a fine needle with a gage ranging between 25-27 G, and/or 27-30 G, and/or between 30 G-33 G.

According to some embodiments, the device may include one or more actuators. Optionally, the device may utilize one or more of various kinds of actuators (for example, spring, and/or elastic member, and/or elastic band, and/or magnetic actuator, and/or electrical actuator).

According to some embodiments, the device may include one or more triggers. Optionally, the device may utilize one or more of various kinds of triggers (for example, hydraulic trigger, and/or lever, and/or button, and/or cable, and/or tension, and/or compression, etc.).

In some embodiments the device and/or various components thereof, may be composed of polyvinyl siloxane (PVS), and/or other plastics, and/or rubber, and/or elastomer, and/or other materials and/or any combination thereof.

The current invention in some of its embodiments may possess some or all of the following advantages: it may be designed to be light-weight, which may contribute to the stability and control of the needle; it may be cheap (for example: disposable); it may be easy to clean and/or sterilize; the device may be mechanical; it may not interfere with the user grip of the proximal end of the syringe (e.g., a user may grip the syringe near the proximal end thereof and/or near the needle to improve stability and control of needle position); it may not interfere with the user seeing movement of the plunger; the device may be extremely stable, allowing for predictable movement, even when using relatively high forces (e.g., to push highly viscous fluids and/or objects through a thin needle; the device may be used with a variety of existing syringes; the user may be able to hold the syringe (e.g., control the needle position) and control rate and/or volume of injection with one hand.

### EXEMPLARY EMBODIMENTS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

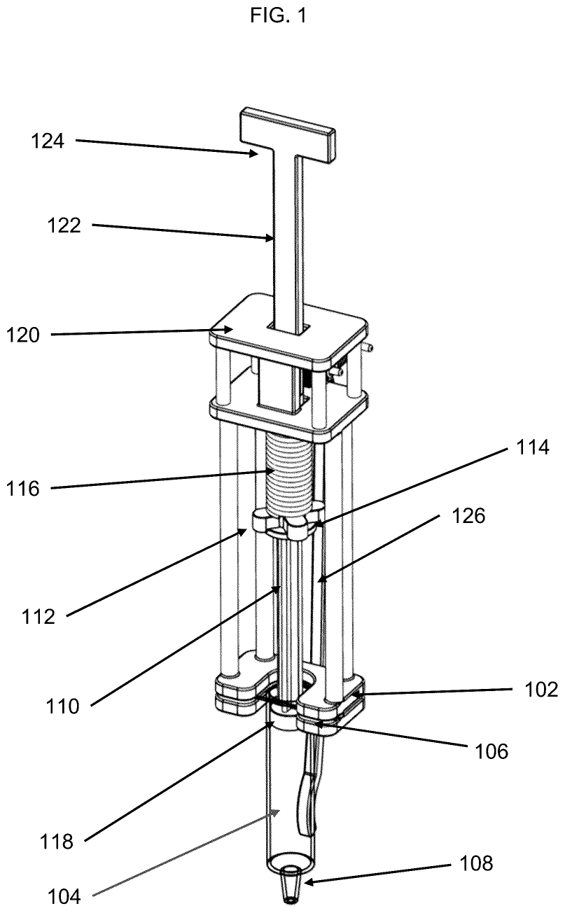

FIG. 1 is a schematic view of a system in accordance with an embodiment of the current invention. For example, in the system, a syringe may be inserted into the device. In some embodiments, the syringe may be held in place by the device's barrel interface 102 gripping the syringe barrel 104. For example, the barrel interface 102 may grip the barrel flange 106 and/or the body of the syringe barrel 104 and/or the needle interface 108 of the syringe barrel. In some embodiments, the syringe plunger 110 may be gripped by the device's plunger interface 112. For example, the plunger interface 112 may include grip a distal portion of the syringe plunger 110 and/or a flange 114 of a plunger of the syringe. Alternatively or additionally, the plunger interface may transfer a force (e.g., pushing) to the plunger without gripping the plunger.

In some embodiments, the device includes a main actuator 116. Optionally, the device may include an actuator and/or a control of the actuator interconnecting between the plunger interface and the barrel interface. Optionally, the main actuator 116 applies a force between the barrel interface 102 and the plunger interface 112 and/or between the barrel 104 of the syringe and the plunger of the syringe. Additionally, and/or alternatively, the actuator 116 and/or plunger interface 112 applies a force on the plunger 110, for example, resulting in a smooth sliding movement between the plunger 110 and the barrel 104 as the plunger seal 118 moves towards the needle interface 108 of the syringe barrel. Optionally, pressure from the main actuator forcing the contents of the syringe and/or into the desired tissue at the desired location. Optionally, the main actuator 116 causes relative movement between the barrel interface 102 and the plunger interface 112 and/or between the barrel 104 of the syringe and the plunger 110 of the syringe. Optionally, the device may include a support structure 120, which may optionally house a kinetic brake pad 122 and/or a trigger activated plunger 124. Optionally, the trigger activated plunger 124 may connect to a trigger 126. Optionally, the trigger 126 may be located on a side of the device, facilitating one handed control of the volume and/or rate of injection. For example, pressure on the trigger may release the brake facilitating movement of the plunger (e.g., facilitating the force of the main actuator pushing the plunger).

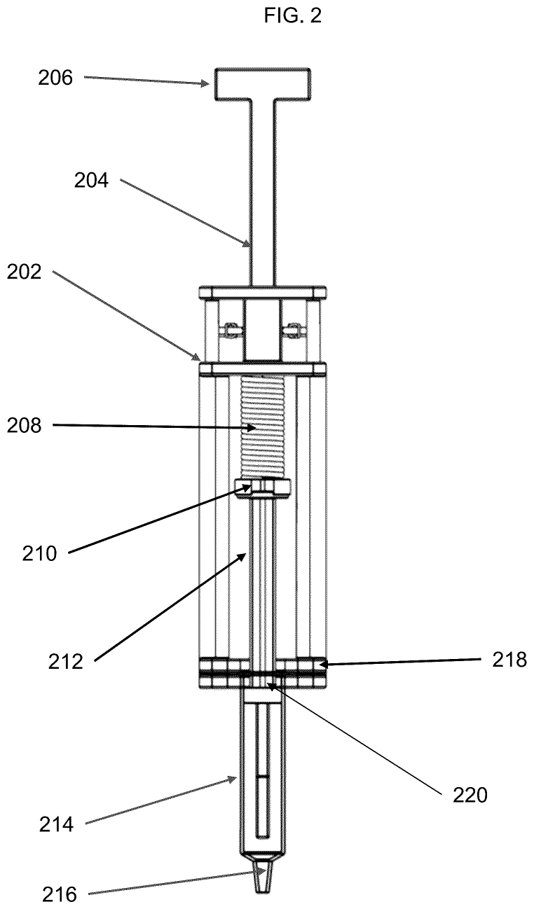

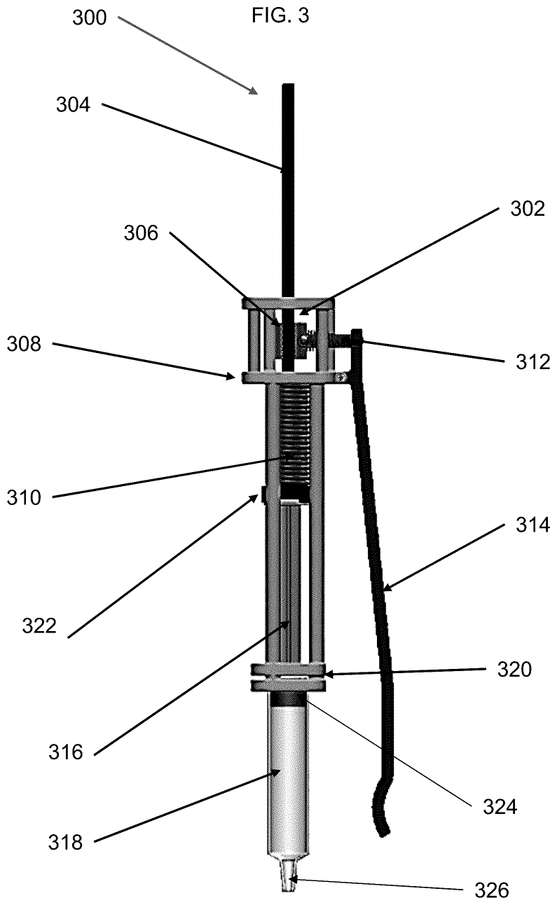

FIG. 2 is a schematic view of a system in accordance with an embodiment of the current invention. For example, in the system, a syringe may be inserted into the device. For example, the device may include a support structure 202 housing a kinetic brake pad 204 and/or a trigger activated plunger 206. Trigger activated plunger 206 may be associated with a main actuator 208, which may be compressed when the device is in the open state prior to use, and uncompressed when the device is in the closed state after to use. The main actuator may be connected to a plunger interface 210 which grips and/or pushes a syringe plunger 212. The syringe plunger 212 is extended away from the needle interface 216 of the syringe barrel 214 when the device is in the open state prior to use, and contained within the syringe barrel 214 close to the needle interface 216 when the device is in the closed state after to use. Syringe barrel 214 is gripped by the barrel interface 218 above the plunger seal 220 of the syringe. FIG. 3 is a schematic view of a system in accordance with an embodiment of the current invention. For example, in the system, a syringe may be inserted into the device. For example, the control system of the device may include a kinetic brake pad 304 e.g., a pad on a trigger activated plunger, between the barrel interface 218 and the plunger interface 210.

As used herein, according to some embodiments, the term “kinetic brake pad” refers to a portion of a brake 302 that moves with respect to the syringe barrel and/or barrel interface and/or moves along with the syringe plunger. As used herein, according to some embodiments, the term “stationary brake pad” refers to the portion of the brake that is stationary in relation to the syringe barrel. For example, the kinetic brake pad 304 may include a linear kinetic brake pad, which may move relative to one or more resistance pads of the static brake pad 306. Optionally, the brake 302 may be supported and/or housed by the support structure 308 Optionally, the actuator 310 remains stationary until it is activated by a user. Optionally, activating may include releasing a stationary brake 306 (e.g., from an engaged state inhibiting movement to disengaged state) facilitating movement of the actuator 310 and/or the kinetic brake pad 304 and/or plunger 316. Optionally, the actuator 310 may include a main actuator including a spring that remains compressed until the user applies pressure to the trigger 314 releasing a brake 302 via the brake/trigger interface 312. Optionally, releasing brake 302 via the brake/trigger release interface 312 may facilitate movement of the kinetic brake pad 304 and/or syringe plunger 316. The user may have gradual and/or graduated and/or continuous control of the braking force of the brake facilitation to gradual and/or graduated and/or continuous control of plunger movement. Optionally, plunger 316 movement may be smooth and/or controlled. Optionally, the device allows a user to predefine a maximum dosage at which point the injection will stop until released and/or reset. Optionally, the trigger activated plunger 300 (e.g., including a kinetic brake pad) may be used to set a spring (e.g., to compress the main actuator for example, by pulling up or depressing the trigger activated plunger).

In some embodiments, a trigger 314 may be actuatable from a proximal portion of a syringe. For example, the trigger may extend from the brake 302 proximally.

In some embodiments of the invention, the barrel of the syringe 318 is held by a barrel interface 320. For example, the barrel interface 320 may clamp to a flange of the barrel and/or to the body of the barrel 318. Optionally, movement of the trigger activated plunger 300 and/or the plunger interface 322 and/or the syringe plunger 316. For example, actuating the trigger 314 may release a braking mechanism 302. For example, releasing the braking mechanism may release the plunger interface 322 to move and/or to push the plunger. Optionally, this facilitates movement of the plunger 316 moving with respect to the syringe barrel 318 and/or barrel interface 320 and/or thereby depressing the syringe plunger 316 and/or depressing the plunger seal 324 towards the needle interface 326, administering the syringe contents.

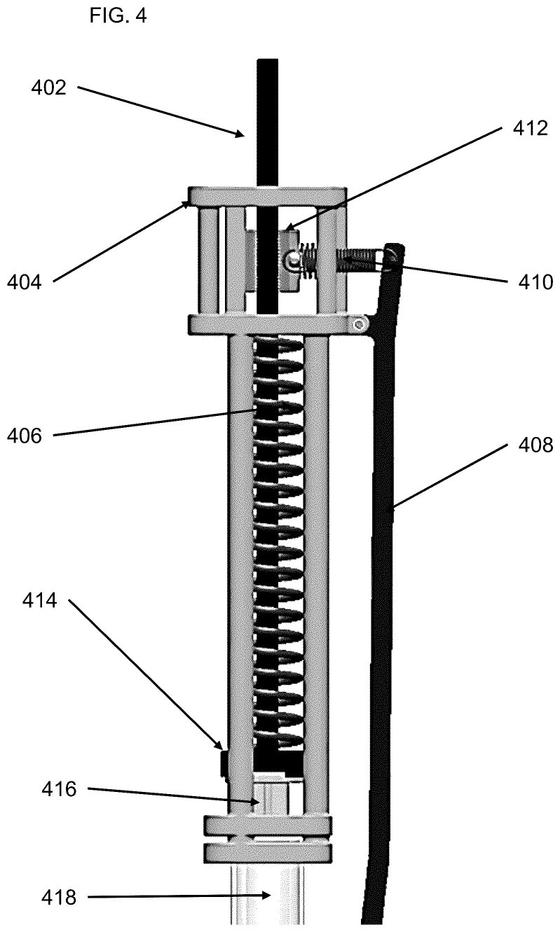

FIG. 4 is a schematic view of a brake system in accordance with an embodiment of the current invention. In this view the main actuator 406 is shown in its optionally extended position. In some embodiments, the user optionally actuates the hand-activated trigger 408. Optionally, the trigger release mechanism 410 releases (e.g., partially and/or fully disengages) the grip of the user-controlled static brake pad 412 mounted to a support structure 404, and/or the kinetic brake pad 402 in accordance with the degree of force the user may apply to the trigger 408. Releasing the brake facilitates movement of the kinetic brake pad 412, the plunger interface 414 and/or plunger 416 with respect to the syringe barrel 418. For example, releasing the brake may allow the main actuator 406 to decompress to its extended state. Decompression of the main actuator 406 may result in descent of the kinetic brake pad 402 and/or plunger interface 414 and/or syringe plunger 416. This may move the syringe plunger 416 through the syringe barrel 418 resulting in the delivery of the contents of the syringe. In some embodiments, the Main Actuator may be spring-loaded. Optionally, a small displacement of the trigger partially disengages the brakes facilitating slow movement of the plunger and/or discharge and progressively more displacement of the trigger facilitates further disengagement of the brakes facilitating faster movement of the plunger and/or discharge.

Alternatively, and/or additionally, other forms of main actuators may be used including a stretched elastic which compresses the syringe plunger when the elastic is released from a stretched state to an unbiased state and/or a linear motor and/or a motor and/or a transmission, etc. and/or any combination thereof.

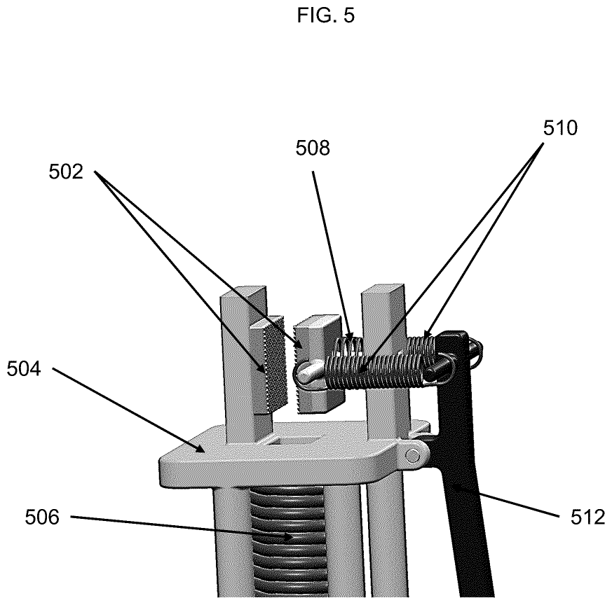

FIG. 5 is a schematic view of a brake system in accordance with an embodiment of the current invention. For example, the device may include one or more trigger biasing actuators 510, which may be biased outward/inward (e.g., by a spring-loaded in some embodiments). Optionally, the brake biasing actuator 508 may be biased to inhibit and/or facilitate movement. In some embodiments, the brake biasing actuator 508 may exert pressure on user-activated and/or resistance static brake pad/s 502 to grip the kinetic brake pad (not shown). The braking system may be mounted to a support structure 504.

Additionally, and/or alternatively, there may be a caliper that also moves or may be static. In some embodiments, at least one static brake pad 502 is stationary. Optionally, at least one static brake pad 502 is user-activated. In some embodiments, the force and/or displacement of a brake pad 502 may be gradually dependent on a force and/or displacement of a trigger 512 from an initial position. Alternatively, and/or additionally, multiple braking structures may be activated differentially depending on the force and/or displacement of the trigger 512 from an initial position. In some embodiments, one brake pad may be made with a high friction resistance surface and one brake pad may be made with a low friction resistance surface. In some embodiments, all the brake pads may have the same resistance. In some embodiments, the trigger biasing actuator 508 and/or actuators 506 may be activated by user hand-pressure on the trigger 512 to effect controlled release of the static brake pad 502 and/or pads. In some embodiments, there may be only one biasing actuator. Optionally, the system is biased to the braking state and/or by actuating the trigger, the brake is released. Optionally, the amount of release and/or the rate of injection gradually increases and/or the rate of movement of the plunger gradually increases as the displacement of the trigger 512.

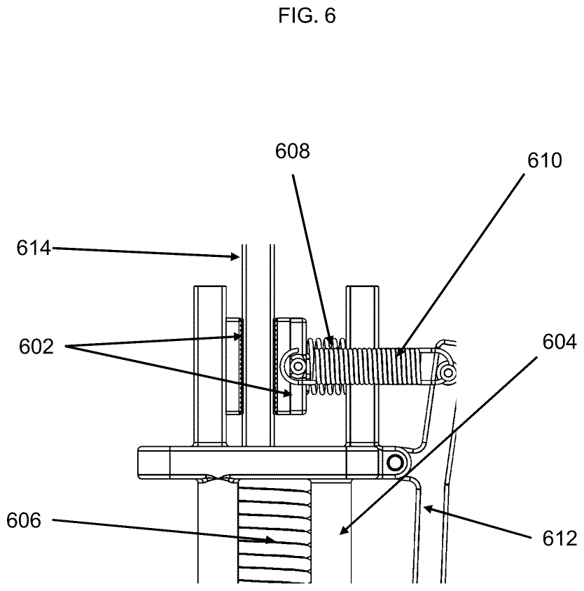

FIG. 6 is a schematic view of a brake system in accordance with an embodiment of the current invention. For example, the device may include one or more trigger biasing actuators 610, which may be biased outward/inward (e.g., by a spring-loaded in some embodiments). Optionally, the brake biasing actuator 608 may be biased to prevent and/or allow movement. In some embodiments, the brake biasing actuator 608 may exert pressure on user-activated and/or resistance static brake pad/s 602 to grip the kinetic brake pad 614. The braking system may be mounted to a support structure 604.

Additionally, and/or alternatively, there may be an actuator 606 that also moves or may be static. In some embodiments, at least one static brake pad 602 is stationary. Optionally, at least one static brake pad 602 is user-activated. In some embodiments, the force and/or displacement of a brake pad 602 may be gradually dependent on a force and/or displacement of a trigger 612 from an initial position. Alternatively, and/or additionally, multiple braking structures may be activated differentially depending on the force and/or displacement of the trigger 612 from an initial position. In some embodiments, one brake pad may be made with a high friction resistance surface and one brake pad may be made with a low friction resistance surface. In some embodiments, all the brake pads may have the same resistance. In some embodiments, the trigger biasing actuator 608 and/or actuators 606 may be activated by user hand-pressure on the trigger 612 to effect controlled release of the static brake pad 602 and/or pads. In some embodiments, there may be only one biasing actuator.

FIG. 7. is a schematic view of a system in accordance with an embodiment of the current invention. For example, in the system, a syringe may be inserted into the device. In some embodiments, the user-activated and/or resistance static brake pads 702 of the device may have structure and/or microstructure: for example, smooth topped micro-pillars and/or micropatterned surface 704 which are able to recover after large deformations while dissipating mechanical energy, thus resembling elastomers. Optionally, the brake pads 702 may have micro-pillars and/or nano-pillars and/or grooves and/or channels and/or micro-textured surfaces. Optionally, the brake pads 702 may be bio-inspired, for example they may be modeled after the gripping feet of climbing tree frogs. Optionally, the micropatterned surface 704 may bio bionic micro-patterned surface made of soft material (e.g., elastomer, etc.). Note that FIGS. 1-7 may depict various views of a single embodiment.

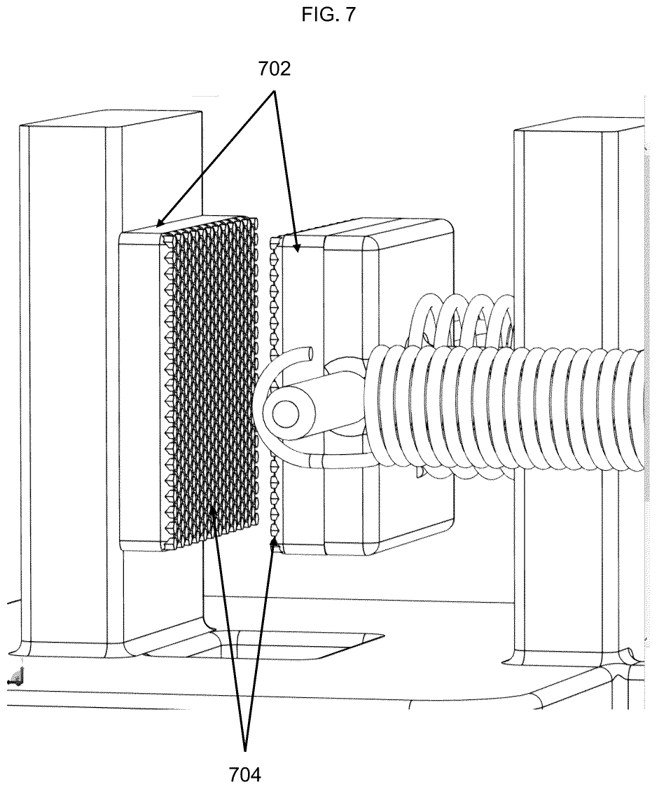

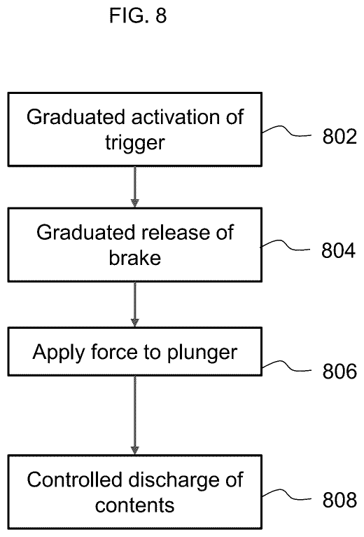

FIG. 8 is a flow chart of a method of syringe injection in accordance with an embodiment of the current invention. For example, in the method, a syringe may be inserted into the device. For example, the syringe may include a preloaded syringe and/or a disposable syringe and/or a conventional syringe configured for manual actuation by hand. In some embodiments, the trigger of the device may be activated 802 in a graduated manner by the user. In response to the activation of the trigger, a brake may be released 804 in a correspondingly graduated manner. Optionally, movement of a syringe plunger may be facilitated in response to the release 804 of the brake. For example, a main actuator may apply force 806 the syringe plunger in a correspondingly graduated manner (e.g., a greater force and/or greater rate of movement in response to a greater displacement of the trigger). Optionally, controlled movement of the plunger may result in the controlled discharge 808 of the contents of the syringe.

In some embodiments, a portion of the trigger and/or a grip with which to hold the syringe may be located near a proximal portion of the syringe and/or near a needle and/or near a discharge location of the syringe, e.g., facilitated simultaneous control of the position of the needle and discharge 808 of the contents of the syringe with one hand.

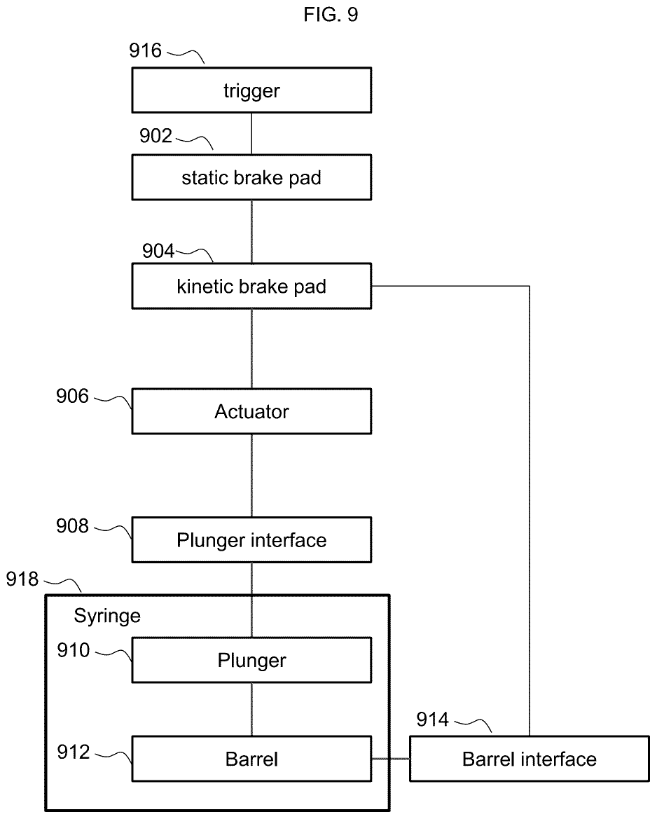

FIG. 9 is a block diagram of a syringe attachment for one-handed automatic injection in accordance with an embodiment of the current invention. For example, the device may include a static brake pad 902 and/or a kinetic brake pad 904. Optionally, one or more of the brake pads 902, 904 is connected to a trigger 916. For example, displacement of the trigger 916 may release the kinetic brake pad 904 from static brake pad 902. Releasing the brakes 902, 904 may facilitate movement of a plunger 910 with respect to a barrel 912 of a syringe 918. For example, releasing the brake 902, 904 may free an actuator 906 connected between a plunger interface 908 and/or a barrel interface 914 to apply a force between the plunger 910 and/or the barrel 912.

In some embodiments, a portion of the trigger and/or a grip with which to hold the syringe may be located near a proximal portion of the syringe and/or near a needle and/or near a discharge location of the syringe, e.g., facilitated simultaneous control of the position of the needle and discharge 808 of the contents of the syringe with one hand.

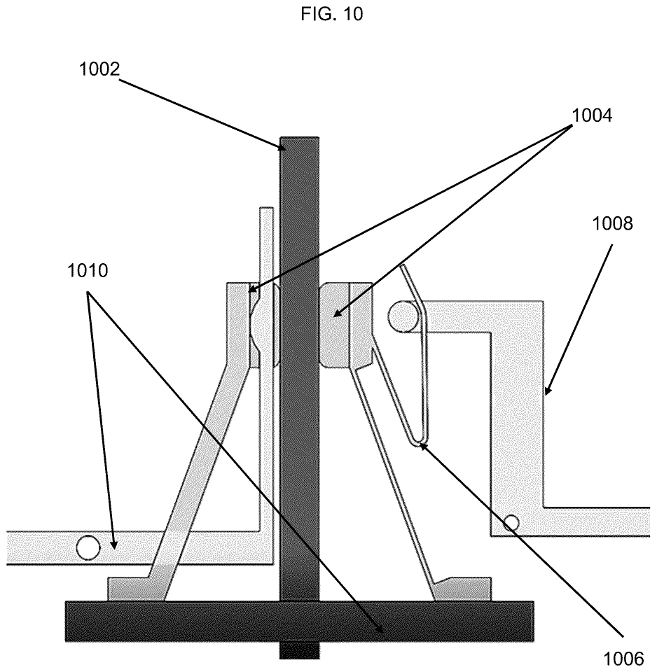

FIG. 10 is a schematic view of a system in accordance with an alternative embodiment of the current invention. In accordance with this embodiment, there may not be a brake biasing actuator. In accordance with this embodiment, there may not be a trigger biasing actuator. In accordance with this embodiment, the user-activated release of the static brake pad/s 1004 may be accomplished via a flat and/or wire torsion spring 1006. The static brake pad/s 1004 may release the kinetic brake pad 1002 when the trigger 1008 is actuated. The static brake pad/s 1004 and/or the flat and/or wire torsion spring 1006 may be attached to a support structure 1010.

In some embodiments, movement of a syringe plunger may be facilitated in response to the release of the brake pads 1004. For example, a main actuator may apply force between the syringe plunger and barrel in a correspondingly graduated manner (e.g., a greater force and/or greater rate of movement in response to a greater displacement of the trigger). Optionally, controlled movement of the plunger may result in the controlled discharge of the contents of the syringe.

In some embodiments, a portion of the trigger and/or a grip with which to hold the syringe may be located near a proximal portion of the syringe and/or near a needle and/or near a discharge location of the syringe, e.g., facilitated simultaneous control of the position of the needle and discharge of the contents of the syringe with one hand.

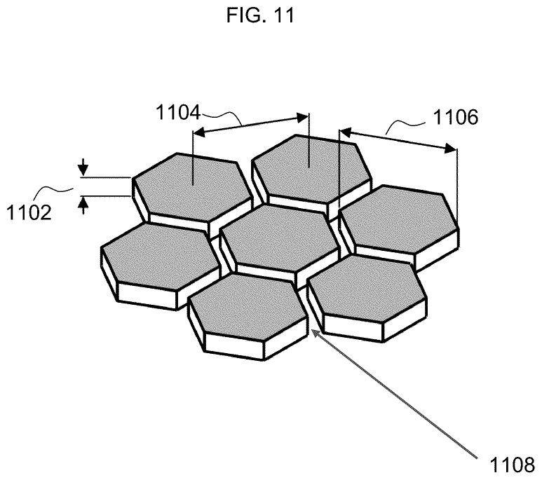

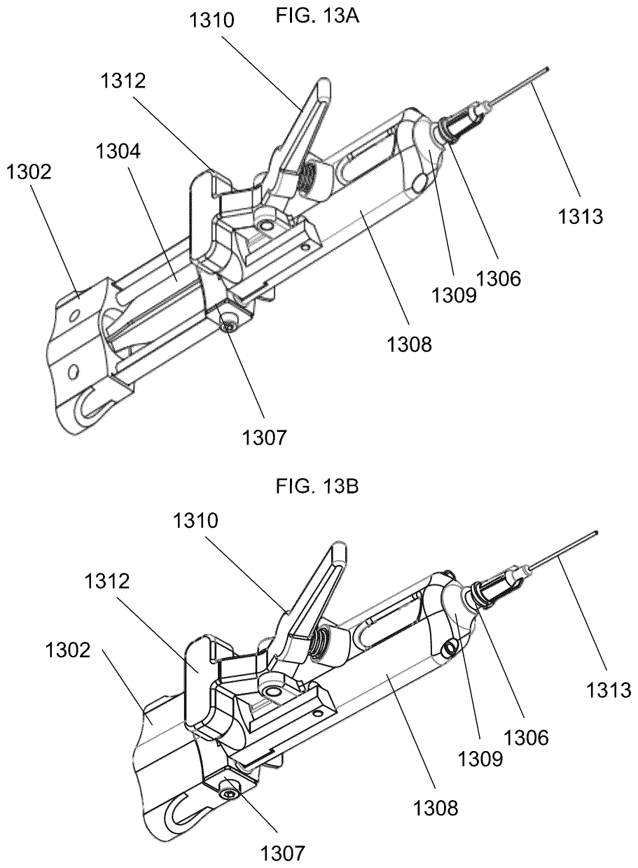

FIG. 11 is a schematic view of a static brake pad of the system in accordance with an embodiment of the current invention. For example, FIG. 11 may illustrate the surface of brake pads 1004, 702, 602, 502, 412. 306 of any of the embodiments of FIGS. 1 to 10 and/or the brake pads 1414, 1508. 1514, 1606 and/or 1616 of embodiments of FIGS. 13 to 16. In some embodiments the static brake pad and/or pads optionally possess a micro-patterned and/or nano-patterned surface. The static brake pad and/or pads may have, for example, smooth topped micro-pillars which are able to recover after large deformations while dissipating mechanical energy, thus resembling elastomers. In some embodiments the pad and/or pads may optionally be made of polyvinyl siloxane (PVS), and/or other elastomers, and/or rubber and/or plastic, and/or other materials. Optionally, the other elastomers may be poly(dimethylsiloxane) (PDMS) and/or polydioxanone (PDO, PDS). Optionally, the soft elastomer may have an elastic modulus ranging between 3-10 MPa, and/or between 4-8 MPa, and/or between 5-7 MPa.

According to some embodiments, the brake pads may be configured for continuous control of friction over a range of velocities (flow rates) to avoid slip-stick disturbances. Optionally the brake pads may have micro-pillars and/or nano-pillars and/or grooves and/or channels 1108. Optionally the brake pads may be bio-inspired, for example they may be modeled after the gripping feet of climbing tree frogs. Optionally, the micro-pillars may have a height 1102 ranging between 0.2-0.5 μm, and/or between 0.5-1 μm, and/or between 1-20 μm, and/or between 20-100 μm, and/or between 100-200 μm and/or between 200-400 μm and/or between 400-800 μm. Optionally, the micro-pillars may have a diameter (for example for example for non-circular pillars the diameter may be defined as twice the hydraulic radius) and/or width 1106 ranging between 10-100 μm, and/or 30-100μm, and/or 20-200μm, and/or between 100-300μm, and/or between 300-600μm, and/or between 600-900μm, and/or between 900-1200μm, and/or between 1200-3000μm.

According to some embodiments, the micro-pillars may have a distance between the mid-points of adjacent pillars 1104 ranging between 50-100 μm, and/or between 100-300 μm, and/or between 300-600 μm, and/or between 300-900 μm, and/or between 900-1200 μm, and/or between 1200-3000 μm.

According to some embodiments, the ratio between the height of the static brake pads to the width of the static brake pads may range between 1:5 to 1:2. According to some embodiments, the ratio between the space between the static brake pads to the width of the static brake pads may range between 1:10 to 1:2.

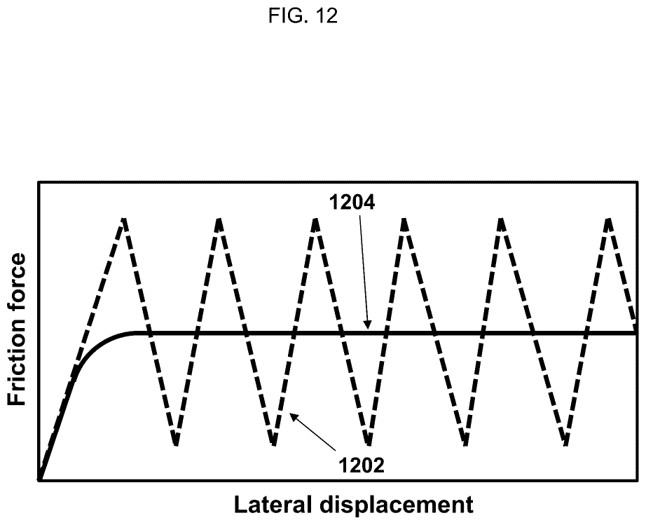

FIG. 12 is a graph indicating a potential advantage which may be gained from micropatterned surfaces. The graph indicates that smooth surfaces may exhibit significant friction instability (e.g., stick-slip disturbances). For example, the stick-slip disturbances may cause the displacement 1202 to jump and stop irregularly. Through the use of special surface, micropatterned surfaces, bionic micropatterned surfaces, bionic controlled slip surface, however, instabilities may be reduced significantly and/or be negligible. This means that bionic micropatterned surface behavior may be significantly more stable and predictable than smooth surface behavior. For example, displacement 1204 may be smooth.

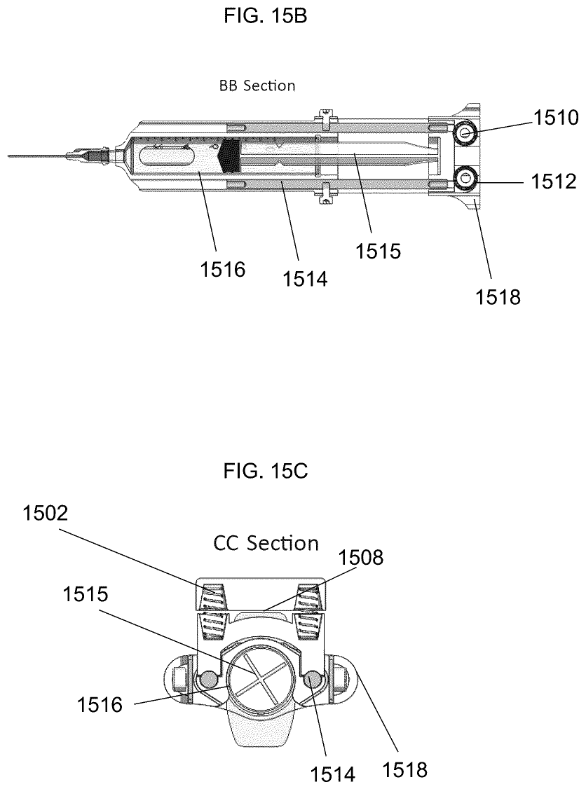

FIGS. 13A and 13B are various schematic views of an open and closed state of the system, respectively, in accordance with an embodiment of the current invention. For example, in the system, a syringe may be inserted into the device. In a first state, (e.g., illustrated in FIG. 13A) a full syringe is loaded into the attachment device. When the syringe is full, the syringe plunger 1304 extends back (distally) away from the proximal discharge portion of the barrel (e.g., the needle interface 1306). Optionally, a plunger interface 1302 is extended distally away from a barrel interface 1308. For example, a distal portion 1307 of the barrel interface 1308 may grasp a flange of the barrel of the syringe and/or a proximal portion 1309 of the barrel interface 1308 may hold back a proximal portion of the barrel of the syringe and/or grasp a needle interface 1306 of the barrel. For example, FIG. 13A may illustrate the system prior to use. Optionally, one or more actuators (for example, coil springs 1510 and 1512 illustrated in FIG. 15B) pull the plunger interface 1302 towards the barrel interface 1308 of the attachment device.

In a second state (e.g., illustrated in FIG. 13B), an empty syringe, (e.g., after to use with the syringe plunger (not shown) inserted fully proximally within the syringe barrel) the plunger interface 1302 has been pulled proximally close to the barrel interface 1308. Transition from the first configuration to the second configuration occurs, for example, when a user actuates a trigger 1310 to release a brake mechanism 1312. Releasing the brake mechanism 1312 facilitates the actuator pulling the plunger interface 1302 towards the barrel interface 1308 and/or pushing the syringe plunger 1304 through the syringe barrel resulting in the delivery of the contents of the syringe. In some embodiments, the trigger 1310 connects to a brake mechanism 1312. Optionally, the brake mechanism 1312 may be location at a distal portion of the barrel interface 1308. Optionally, the trigger 1310 extends proximally to a proximal towards a proximal portion 1309 of the barrel interface 1308. For example, a user may use one hand to hold the proximal portion 1309 of the barrel interface 1308 (for example to position a needle 1313 of the syringe) while actuating the trigger 1310.

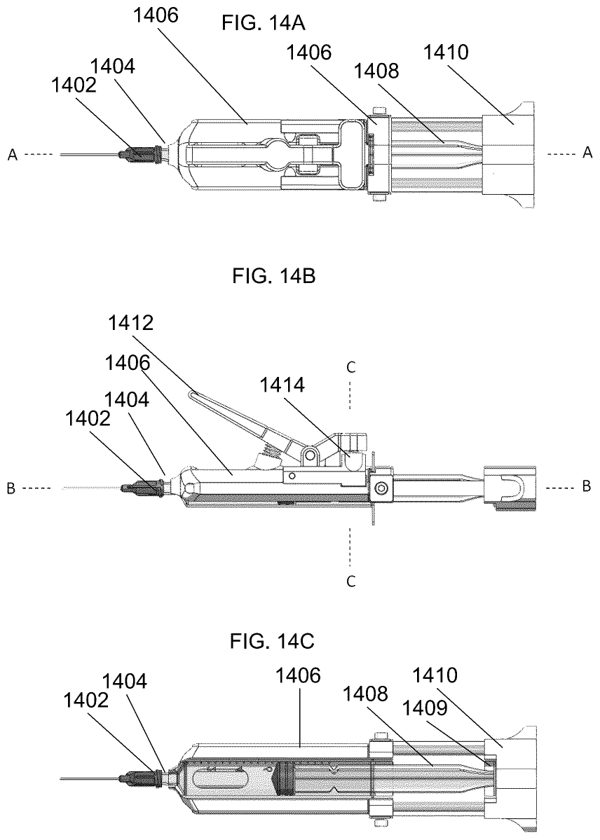

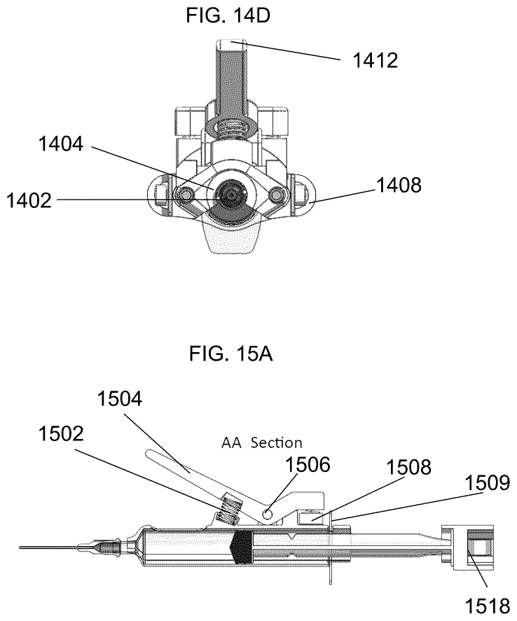

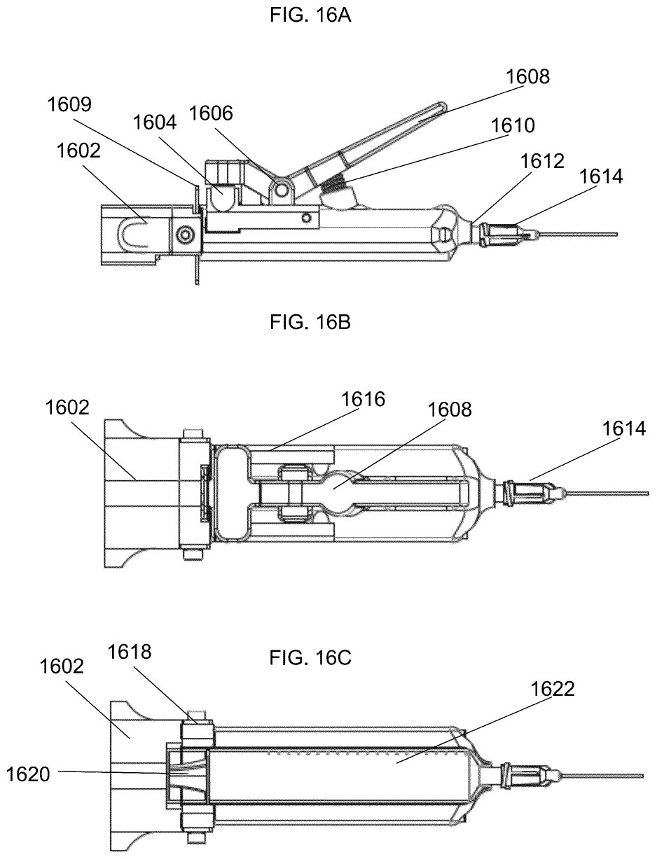

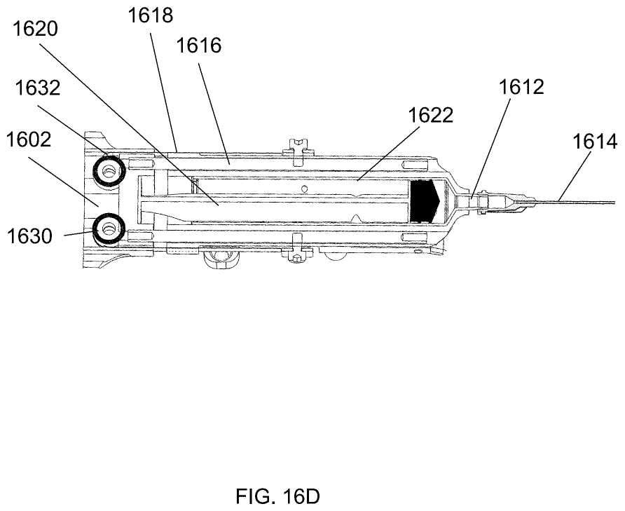

FIGS. 14A-14D are a schematic top view, side view, bottom view and front axial view, respectively, of an open state system in accordance with an embodiment of the current invention, for example, it may be the same as the system of FIGS. 13A and 13B. For example, in the system, a syringe may be inserted into the device. For example, FIGS. 14A, 14C and 14D are a top view, a bottom view, and front axial view, respectively, of a syringe inserted into the device. The syringe is gripped by a syringe barrel interface 1406 and/or a plunger interface 1410. The interfaces 1406, 1410 may grip and/or retain the syringe around the syringe barrel and/or on a barrel flange (for example flange 1509 of FIG. 15A and/or flange 1609 of FIG. 16B) and/or at the needle interface 1404. The plunger interface 1410 may push and/or grip the plunger 1408 and/or plunger flange 1409.

According to some embodiments, the syringe may include a fine needle 1402 attached to the needle interface 1404 of the syringe. In some embodiments, the needle may have a gauge ranging between 25-27 G, and/or 27-30 G, and/or between 30G-33 G.

According to some embodiments, the device may include one or more brake pads 1414 controlled by a trigger 1412. Optionally, the trigger 1412 may be actuated to release a brake pad. For example, releasing a brake pad 1414 may facilitate depressing the plunger and/or injecting the contents of the syringe. Optionally, the device may facilitate holding the syringe and/or controlling a needle position. Optionally, the device may facilitate controlling the rate and/or volume of injection with one hand. For example, the device may facilitate holding the syringe near a proximal end thereof and/or the device may facilitate gradual control over a rate of discharge of the syringe from a proximal portion of the syringe. For example, a user may hold a proximal portion of the device of FIGS. 14A-14D between some of the fingers and the thumb of one hand and simultaneously press the trigger with a free finger of that hand. Alternatively or additionally, a user may hold a proximal portion of the device of FIGS. 14A-14D with the fingers of one hand and simultaneously press the trigger with the thumb of that hand.

The user may have gradual and/or graduated and/or continuous control of the braking force of the brake facilitation to gradual and/or graduated and/or continuous control of plunger movement. Optionally, plunger movement may be smooth and/or controlled. Optionally, the device may allow a user to predefine a maximum dosage at which point the injection will stop until released and/or reset.

FIGS. 15A-15C are various schematic views of an open state system in accordance with an embodiment of the current invention. For example, FIGS. 15A-15C are further views of the system of FIGS. 13A-14D. For example, in the system, a syringe may be inserted into the attachment device. For example, in FIG. 15A, the device may include a trigger mechanism which may include a handle 1504 connected by a brake biasing actuator 1502 (e.g., a compression spring, etc.) to a support structure. The trigger handle 1504 may include a fulcrum 1506 about which the handle may be rotated. The handle 1504 may be connected to dual static brake pads 1508. Optionally, when the handle 1504 is depressed, the handle 1504 may rotate about the fulcrum 1506 to release pressure between the dual static brake pads 1508 and one or more kinetic brake pads (e.g., kinetic brake pads 1514 illustrated in FIG. 15B). Optionally, the kinetic brake pad 1514 is positioned adjacent to (e.g., beside alternatively or additionally above and/or below) the syringe plunger. The kinetic brake pad 1616 optionally travels along with and/or parallel to the plunger 1620. For example, in the open state when the plunger is positioned behind the syringe barrel, the kinetic brake pad 1514 may also be positioned behind and/or adjacent to (e.g., beside, above, and/or below) the syringe barrel. Optionally, the dual static brake pads may include micro-patterned surfaces, for example as illustrated in FIG. 11.

Advantageously, a large movement of the handle may result in a small movement of the static dual brake pads, thereby providing good control of the rate and/or volume injected. For example, the brake is gradual therefore, small movement of the trigger result in à slow discharge rate, while large pressure and/or movement of the trigger results in à fast discharge rate.

FIG. 15B is a BB cross section of a schematic view of the system in accordance with some embodiments. For example, the device may include a main actuator, e.g., a tension spring, for example a pair of torsion coil springs 1510 and 1512. Optionally, the main actuator may be associated with one or more balanced guides e.g., bars, and/or one or more kinetic brake pads 1514. Optionally, the kinetic brake pads 1514 may be located on the sides of the device (e.g., adjacent to the syringe plunger 1516 and/or in front of the plunger interface (e.g., as illustrated in the embodiment of FIG. 15B)). Alternatively or additionally, the kinetic brake pads may be located behind the syringe plunger and/or behind the plunger interface (e.g., as illustrated in FIGS. 1-7). Optionally, when the kinetic brake pad 1515 is located on the sides of the device and/or in front of the plunger interface 1518, the device may be shorter than when the kinetic brake pad is located behind the plunger interface. Optionally, such a configuration of the kinetic brake pad may be easier to use with one hand.

FIGS. 16A, 16B, 16C and 16D are schematic side, top, bottom and cross-sectional views, respectively, of a closed state system in accordance with an embodiment of the current invention. For example, in the system, a syringe may be inserted into the device. For example, in a second configuration of the device, the device may include a trigger mechanism which may include a handle 1608 connected by a brake biasing actuator 1610 (e.g., a compression spring, etc.) to a support structure. The trigger handle 1608 may include a fulcrum 1606 about which the handle 1608 may be rotated. The handle 1608 may be connected to dual static brake pads 1604 and/or a main actuator (not shown). The main actuator may be associated with one or more kinetic brake pads 1616. Optionally, the kinetic brake pads 1616 may be located on the sides of the device and/or above or below the device. For example, in the closed state the kinetic brake pads 1616 are located adjacent to the syringe plunger and/or adjacent to the syringe barrel 1622 and/or in front of the plunger interface 1602. The syringe may be gripped by one or more syringe interfaces 1618, and/or a plunger interface 1602. Optionally, when the handle 1608 is depressed, the handle 1608 may rotate about the fulcrum 1606 to release the dual static brake pads 1604. The syringe plunger 1620 gripped by the plunger interface 1602 is compressed within the syringe barrel 1622 close to the needle interface 1612 resulting in the delivery of the contents of the syringe through the needle 1614. The main actuator of the device optionally includes a pair or torsion springs 1630 and 1632.

It is expected that during the life of a patent maturing from this application many relevant technologies will be developed, and the scope of the terms are intended to include all such new technologies a priori.

As used herein the term “about” refers to ±10 %

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A system for one-handed injection comprising:
a syringe including a plunger and a barrel; and
a syringe attachment device, wherein the syringe is coupled to the syringe attachment device; and wherein the syringe attachment device comprises:
a syringe interface configured to grip the barrel of the syringe;
a main actuator configured to apply force between the syringe interface and the plunger;
a braking system configured for inhibiting movement of the plunger;
a trigger configured to graduate activation of said braking system;
wherein the system is configured for simultaneously controlling a needle position and for actuating said trigger with one hand.

2. The system of claim 1, further comprising a plunger interface interconnecting between said main actuator and said plunger and wherein said inhibiting movement of the plunger is by said braking system inhibiting movement of said plunger interface.

3. The system of claim 2, wherein said plunger interface grasps said plunger.

4. The system of claim 3, wherein said plunger interface grasps a flange of said plunger.

5. The system of claim 1, wherein said syringe interface grasps a flange of said barrel of the syringe.

6. The system according to claim 1, wherein the braking system comprises a static brake pad and at least one kinetic brake pad.

7. The system according to claim 6, wherein at least one of said static brake pad and said kinetic brake pad comprises a micro-patterned brake pad.

8. The system according to claim 6, wherein at least one of said static brake pad and said kinetic brake pad is biomimetic.

9. The system according to claim 7, wherein the micro-patterned brake pad is composed at least in part by an elastomer.

10. The system according to claim 9, wherein the elastomer has an elastic modulus ranging between 3-10 MPa.

11. The system according to claim 10, wherein the elastomer includes poly(dimethylsiloxane) (PDMS), polydioxanone (PDO, PDS) or a combination thereof.

12. The system according to claim 1, wherein the braking system is biased to prevent limit movement of said plunger and wherein actuating said trigger releases said braking system in a graduated manner to facilitate graduated movement of the plunger with respect to the barrel.

13. The system according to claim 12, wherein a predefined maximum rate of injection is set by the trigger, the braking system, the main actuator or any combination thereof.

14. The system according to claim 1, wherein the trigger is configured to release the braking system permitting the main actuator to depress the plunger.

15. The system according to claim 14, wherein a braking force is controlled by the trigger.

16. The system according to claim 15, wherein the braking force is released gradually according to a displacement of said trigger to facilitate gradual increase in rate of movement of the plunger in response to gradual increase of displacement on the trigger.

17. The system according to claim 1, wherein the syringe interface is configured to grip a syringe barrel, a needle interface, or both.

18. The system according to claim 1, wherein the device is lightweight.

19. The system according to claim 1, wherein the device is disposable.

20. The system according to claim 1, wherein the device is easy to clean and to sterilize.

21. A method of using a syringe attachment for one-handed injections, the method comprising:
supplying a syringe in a syringe attachment device with a main actuator configured to apply a force between a plunger of said syringe and a barrel of said syringe and said attachment device further including a braking system inhibiting movement of said plunger with respect to said barrel;
activating a trigger of the device thereby releasing a braking system resulting in discharge of the syringe.

22. The method according to claim 21, further comprising increasing a rate discharge of the syringe by increasing a level of activation of said trigger.

23. The method according to claim 22, further comprising simultaneously controlling a position of a needle of the syringe and said activating with one hand.

24. The method according to claim 21, further comprising simultaneously controlling a position of a needle of the syringe and said activating with one hand.
